(19) United States
(12) Patent Application Publication
(10) Pub. No.: US 2006/0165561 A1
(43) Pub. Date: Jul. 27, 2006

(54) Apparatus and method to calibrate a gas detector

(76) Inventor: David A. Rohrbacker, Tucson, AZ (US)

Correspondence Address: DALE F. REGELMAN, LAW OFFICE OF DALE F. REGELMAN, P.C., 4231 SOUTH FREMONT AVENUE, TUCSON, AZ, 85714, US

(21) Appl. No.: 10/524,218

(22) PCT Filed: Oct. 31, 2003
(86) PCT No.: PCT/US03/34651

§ 371 Date: Feb. 8, 2005

Related U.S. Application Data
(60) Provisional application No. 60/422,667, filed on Oct. 31, 2002.

(51) Int. Cl. G01N 33/00

(52) U.S. Cl. 422/68.1

(57) ABSTRACT

An analyte chamber which can be releaseably attached to a portable calibration apparatus. The analyte chamber include a wick, one or more analytes disposed in that wick, a housing having an open end, where the wick is internally disposed within that housing, and at least one layer disposed over and enclosing the open end. The analyte chamber does not include a releaseable quantity of the liquid phase of the one more more analytes absorbed in the wick. A method to calibrate an immobile gas detector. The method provides a portable calibration apparatus comprising a portable detector and an analyte chamber comprising a wick, where the analyte chamber is capable of providing one or more concentrations of one or more gases to the portable detector, and where the portable calibration apparatus is capable of providing those one more more gases to the immobile gas detector.

### FIELD OF THE INVENTION

This invention relates to a chamber for one or more analytes, where the one or more analytes are absorbed in a wick disposed within the chamber. The invention further relates to a portable calibration apparatus which includes Applicant's analyte chamber, and a method using that portable calibration apparatus.

### BACKGROUND OF THE INVENTION

Gas detectors find use in many industrial processes. Many such gas detectors measure the concentration of one or more substances, i.e. analytes. Many times the one or more gaseous analytes are flammable and/or toxic. Many of these gas detector systems are “hard plumbed” into certain process equipment, and therefore, are not portable.

In order to accurately measure the concentration of one or more gases flowing through the process equipment, the gas detector must periodically be calibrated. Such calibration requires that the detector be provided with a known concentration of a known gas. The detector's measurement system is then adjusted to reflect the known concentration of the known gas.

Because the process gas detector is not portable, that detector must necessarily be calibrated “in the field.” Thus, a portable calibration apparatus which can supply a known concentration of a known gaseous analyte must be brought to the process gas detector. In certain prior art systems, the gas supply for such a portable calibration apparatus comprises a gas stored under pressure in a cylinder. Transportation of such pressurized cylinders is sometimes troublesome, and is likely regulated by numerous federal/state transportation regulations.

Other prior art portable calibration units include a macroscopic quantity of a volatile liquid(s) which can vaporize and provide a supply of one or more gases, for the portable calibration apparatus. Once again, use of these prior art systems can be problematic. The volatile liquid may be flammable and/or toxic. Such volatile liquids must be transported in a suitable, leak-proof container. In the event that container is dropped or impacted, however, a release of a flammable/toxic liquid will occur. Such a release could present a fire and/or toxicity situation requiring the response of a fire department and/or hazmat remediation unit. For these reasons, transport of such a quantity of a volatile liquid presents problems.

What is needed, is an apparatus and a method to provide one or more gaseous analytes in combination with a portable gas detector calibration unit, where that portable gas calibration detector unit does not include one or more pressurized gases, one or more releaseable volatile liquids, or combinations thereof. Applicant's invention provides such an apparatus and method.

### SUMMARY OF THE INVENTION

Applicants' invention includes an analyte chamber which can be releaseably attached to a portable calibration apparatus. Applicant's analyte chamber includes a wick, one or more analytes disposed in that wick, a housing having an open end, where the wick is internally disposed within that housing, and at least one layer disposed over and enclosing the open end. Applicant's analyte chamber does not include a releaseable quantity of the liquid phase of the one or more analytes absorbed in the wick.

Applicant's invention further includes a method to calibrate an immobile gas detector. Applicant's method provides a portable calibration apparatus comprising a portable detector and an analyte chamber comprising a wick, where the analyte chamber is capable of providing one or more concentrations of one or more gases to the portable detector, and where the portable calibration apparatus is capable of providing those one or more gases to the immobile gas detector.

One or more analytes are disposed by capillary action in the wick. Applicant's method then provides from the wick to the portable detector one or more concentrations of the one or more analytes in the gaseous phase. The portable detector measures those one or more concentrations as Applicant's method provides the one or more gaseous analytes to the immobile detector. The immobile detector is then calibrated using the measured one or more concentrations, i.e. the immobile detector is adjusted to recite the same concentration(s) as does the portable detector.

### BRIEF DESCRIPTION OF THE DRAWINGS

The invention will be better understood from a reading of the following detailed description taken in conjunction with the drawings in which like reference designators are used to designate like elements, and in which:

FIG. 1 is a cross sectional view of a first embodiment of Applicant's analyte chamber;

FIG. 2 is a cross sectional view of a second embodiment of Applicant's analyte chamber;

FIG. 3A is a top view of a mechanical iris defining a first aperture;

FIG. 3B is a top view of a mechanical iris defining a second aperture;

FIG. 3C is a top view of a mechanical iris defining a third aperture;

FIG. 4 is a block diagram of a first embodiment of Applicant's portable calibration apparatus;

FIG. 5 is a block diagram of a second embodiment of Applicant's portable calibration apparatus;

FIG. 6 is a flow chart summarizing the steps of Applicants' method; and

FIG. 7 is a block diagram of a third embodiment of Applicant's portable calibration apparatus;

FIG. 8 is a perspective view of a portable apparatus which includes Applicant's analyte chamber;

FIG. 9 is a graph showing the flow rate of analyte from the apparatus of FIG. 4 or 5 as a function of the frequency of an air pump comprising a positive pressure assembly;

FIG. 10 is a cross sectional view of a third embodiment of Applicant's analyte chamber

### DETAILED DESCRIPTION OF THE PREFERRED EMBODIMENTS

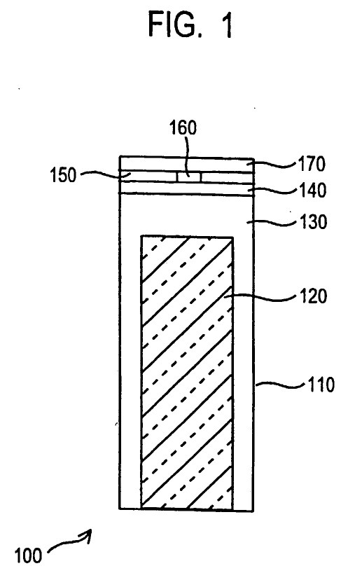

This invention is described in preferred embodiments in the following description with reference to the Figures, in which like numbers represent the same or similar elements. FIG. 1 shows a embodiment 100 of Applicant's analyte chamber. Analyte chamber 100 includes housing 110 and wick 120 internally disposed within housing 110. Housing 110 can be formed from any rigid material that is compatible with the analytes/solvents absorbed in wick 120. Such rigid materials include metal, plastic, and glass. By “glass,” Applicant means an optically clear, non-crystalline material below its glass transitions temperature.

By “wick,” Applicant means a material that can absorb one or more fluids by capillary action. Capillary effects are observed when fluid adhesion to a solid surface produces a force that is sufficient to support the weight of the column of fluid. For example, if a fluid wets the surface of a small capillary tube, it is drawn up until the weight of the column equals the adhesion force.

In certain embodiments, wick 120 is formed from one or more naturally-occurring materials, such as cellulose, cotton, and the like. In certain embodiments, wick 120 is formed from one or more synthetic polymeric materials, including without limitation polytetrafluoroethylene, polyethylene, polypropylene, and the like, and combinations thereof. In certain embodiments, wick 120 is formed from one or more ceramic materials, including without limitation, alumina, mullite, zirconia, and the like, and combinations thereof.

In certain embodiments, wick 120 comprises a woven structure formed from a plurality of individual fibers. In certain embodiments, wick 120 comprises a cellular material formed from a plurality of struts in combination with a plurality of cells. In certain embodiments, such a cellular material includes an open cell foam. In certain embodiments, such a cellular material includes a combination of open-cell and closed-cell structures. In certain embodiments, wick 120 comprises a carbon foam comprising a plurality of pores. The average size of this plurality of pores can be adjusted to enhance the absorbance of a given analyte.

In certain embodiments, a single liquid analyte is disposed internally within wick 120. By “liquid analyte”, Applicant means an analyte that is in the liquid phase at room temperature, or a solid analyte dissolved in one or more solvents. The analyte, and optionally the one or more solvents, are disposed in wick 120 by capillary action such that chamber 100 includes no visibly distinct liquid components. Moreover, the analyte, with or without solvent(s), is absorbed in wick 120 such that wick 120 will not release or emit a liquid fraction of that analyte and/or solvent(s). Therefore, in the unlikely event housing 110 is ruptured, a release of liquid analyte, with or without solvent(s), will not occur.

Housing 110 includes an open end. One or more layers/devices are disposed over, and enclose, that open end. For example, analyte chamber 100 includes semi-permeable layer 140, impermeable layer 150 which is formed to include orifice 160, and impermeable layer 170. Chamber 100 further includes head space 130, where head space 130 comprises the interior volume of housing 110 that is not occupied by wick 120. When impermeable layer 170 is in place, chamber 100 comprises a closed vessel. In that closed vessel, an equilibrium exists between the gaseous concentration of the analyte in head space 130 and the analyte absorbed in wick 120.

The thickness and composition of semi-permeable layer 140 is selected such that a desired gaseous concentration of the analyte will diffuse through layer 140. When impermeable layer 170 is removed, a portion of the gaseous component of the analyte passing through layer 140 will pass through orifice 160 and be released. The rate of release of the analyte from wick 120, through layer 140, and out orifice 160 is a function of, inter alia, the temperature of chamber 110, the thickness of layer 140, the chemical composition of layer 140, and the size of orifice 160.

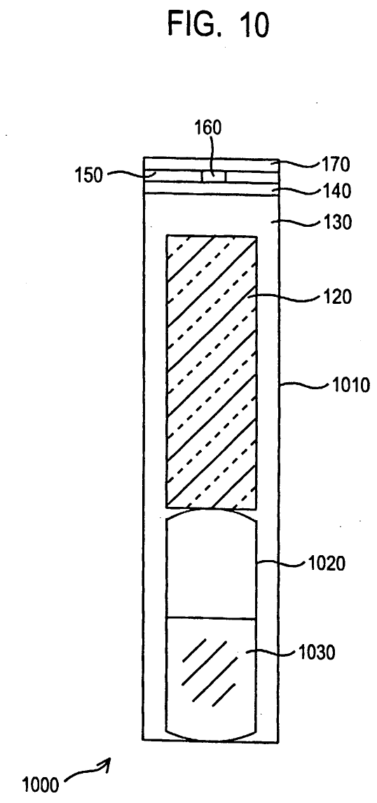

Referring now to FIG. 10, in certain embodiments Applicants' analyte chamber 1000 includes housing 1010, wick 120, and crushable ampoule 1020 which contains analyte/analyte solution 1030. Housing 1010 includes an open end. One or more layers/devices are disposed over, and enclose, that open end. For example, analyte chamber 1000 includes semi-permeable layer 140, impermeable layer 150 which is formed to include orifice 160, and impermeable layer 170. Chamber 1000 further includes head space 130, where head space 130 comprises the interior volume of housing 110 that is not occupied by wick 120 or ampoule 1020. When impermeable layer 170 is in place, chamber 100 comprises a closed vessel. In that closed vessel, and after crushing ampoule 1030, an equilibrium exists between the gaseous concentration of the analyte in head space 130 and the analyte absorbed in wick 120.

The thickness and composition of semi-permeable layer 140 is selected such that a desired gaseous concentration of the analyte will diffuse through layer 140. When impermeable layer 170 is removed, a portion of the gaseous component of the analyte passing through layer 140 will pass through orifice 160 and be released. The rate of release of the analyte from wick 120, through layer 140, and out orifice 160 is a function of, inter alia, the temperature of chamber 110, the thickness of layer 140, the chemical composition of layer 140, and the size of orifice 160.

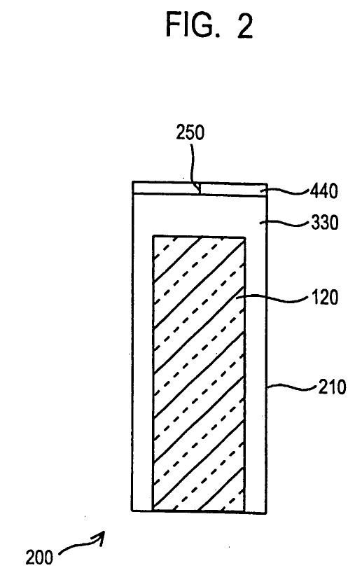

Referring now to FIG. 2, analyte chamber 200 includes housing 210 and wick 120 internally disposed within housing 210. Wick 120 is described above. One or more analytes, with or without one or more solvents, are absorbed in wick 120 by capillary action.

Chamber 200 further includes head space 230. Chamber 200 further includes adjustable orifice assembly 240. In certain embodiments, adjustable orifice assembly 240 comprises an electromechanical assembly. Assembly 240 includes orifice 250, such that the size of orifice 250 can be increased or decreased. In certain embodiments, assembly 240 is formed such that the dimensions of orifice 250 can be adjusted mechanically. When orifice 250 is closed, an equilibrium concentration of gaseous analyte is formed in head space 230. In certain embodiments, assembly 240 is formed such that the dimensions of orifice 250 can be adjusted electrically. In certain embodiments, chamber 200 further includes a crushable ampoule containing an analyte/analyte solution, such as ampoule 1020 (FIG. 10) which contains analyte/analyte solution 1030 (FIG. 10).

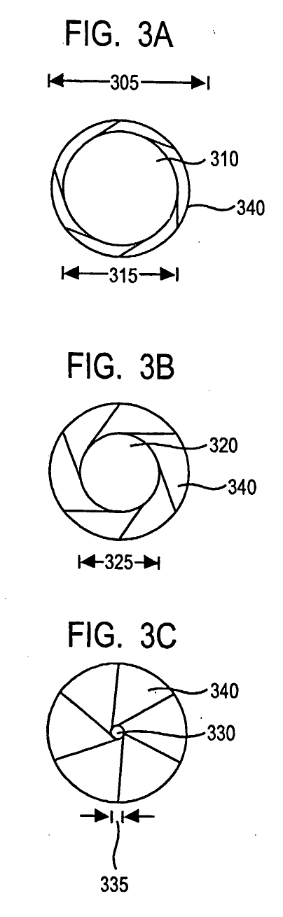

FIGS. 3A, 3B, and 3C, show mechanical iris 340 and orifice 310, 320, and 330, respectively. In certain embodiments, adjustable orifice assembly 240 comprises mechanical iris 340. Assembly 340 has a diameter 305. In FIG. 3A, assembly 340 is adjusted such that orifice 310 has diameter 315, where diameter 315 is about 90 percent or more of diameter 305. In FIG. 3B, assembly 340 is adjusted such that orifice 320 has diameter 325, where diameter 325 is about 50 percent of diameter 305. In FIG. 3C, assembly 340 is adjusted such that orifice 330 has diameter 335, where diameter 335 is about 10 percent or less of diameter 305. Assembly 340 can be further adjusted such no orifice exists therein.

Referring again to FIG. 2, the rate of release of gaseous analyte(s) from wick 120 can be controlled by, inter alia, adjusting the size of orifice 250. As those skilled in the art will appreciate, as the size of orifice 250 increases, the rate of release of gaseous analyte from wick 120 increases. On the other hand, as the size of orifice 250 decreases, the rate of release of gaseous analyte from wick 120 decreases.

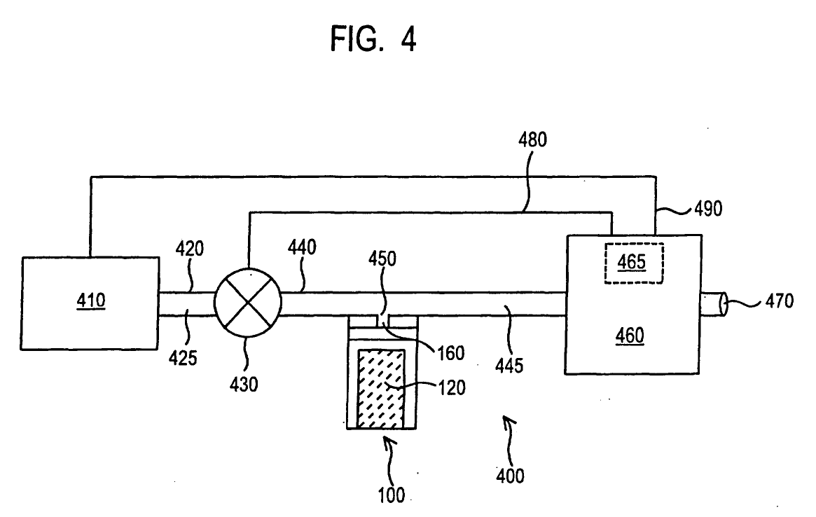

FIG. 4 shows one embodiment of Applicant's calibration apparatus. The embodiment of FIG. 4 includes analyte chamber 100/1000, positive pressure assembly 410, conduits 420 and 440, valve 430, detector unit 460, and conduit 470. Conduit 420 is disposed between positive pressure assembly 410 and value 430. Conduit 420 includes internal passage 425. Positive fluid pressure generated by positive pressure assembly 410 is provided to valve 430 by conduit 420. In certain embodiments, positive pressure assembly 410 provides a fluid at a pressure greater than ambient atmospheric pressure. In certain embodiments, positive pressure assembly 410 provides air at a pressure greater than ambient atmospheric pressure.

Conduit 420 is formed from a rigid material, a flexible material, or combinations thereof, having sufficient mechanical strength to contain internal pressures up to about 120 PSI continuously and up to about 300 PSI for durations of about 30 seconds or less. In certain embodiments, conduit is formed from metal tubing. In certain embodiments, conduit 420 is formed from a polymeric material. In certain embodiments, conduit 420 is formed from an elastomer. By “elastomer,” Applicant means a natural or synthetic material having a glass transitions temperature of less than about −30° C., and an elongation at room temperature of 300% or more. In certain embodiments, conduit 420 is formed from glass.

Conduit 440 is disposed between value 430 and detector 460. Conduit includes internal passage 445. Positive fluid pressure generated by positive pressure assembly 410 is provided to conduit 440 via valve 430. Conduit 440 is formed from a rigid material, a flexible material, or combinations thereof, having sufficient mechanical strength to contain internal pressures up to about 3 atmospheres continuously and up to about 6 atmospheres for durations of about 30 seconds or less. In certain embodiments, conduit 440 is formed from metal tubing. In certain embodiments, conduit 440 is formed from a polymeric material. In certain embodiments, conduit 440 is formed from an elastomer. In certain embodiments, conduit 440 is formed from glass.

Conduit 440 is formed to include orifice 450. In apparatus 400, analyte chamber 100/1000 is disposed adjacent 440 such that orifice 160 communicates with orifice 450. Referring now to FIGS. 1 and 4, prior to removeably affixing chamber 100/1000 to conduit 440, impermeable layer 170 is first removed from chamber 100/1000. Thus, gaseous analyte(s) can diffuse through semi-permeable layer 140, and pass through orifices 160 and 450, and enter into conduit 440. A positive pressure in conduit 440, provided by positive pressure assembly 410 and valve 430, carries that gaseous analyte(s) into detector 460, and to external orifice 470.

Prior to affixing chamber 100/1000 to apparatus 400, detector 460 is calibrated using known concentrations of one or more analytes. After chamber 100/1000 is removeably affixed to apparatus 400, the concentration of the one or more analytes released from analyte chamber 100/1000 is controlled by, inter alia, the fluid pressure provided by positive pressure assembly 410 and valve 430. Feedback loop 480 connects detector 460 and valve 430. Detector 460 measures the concentration of the analyte passing through orifice 470.

Detector 460 includes microprocessor 465. In certain embodiments, microprocessor 465 comprises computer hardware and software to operate detector 460, valve 430, and positive pressure assembly 410. In certain embodiments, microprocessor 465 comprises an Application Specific Integrated Circuit embodying program steps encoded into firmware to operate detector 460, valve 430, and positive pressure assembly 410. Feedback circuit 480 interconnects valve 430 and microprocessor 465. Feedback circuit 490 interconnects positive pressure assembly 410 and microprocessor 465.

As those skilled in the art will appreciate, an equilibrium concentration of gaseous analyte is established within air flow conduit 440 prior to operation of assembly 410 and/or opening of valve 430. When air flows through conduit 440, gaseous analyte is swept out of conduit 440, into detector 460, and out orifice 470. As the equilibrium concentration of gaseous analyte in conduit 450 is perturbed, in accord with LeChatelier's principle, additional analyte is released from wick 120.

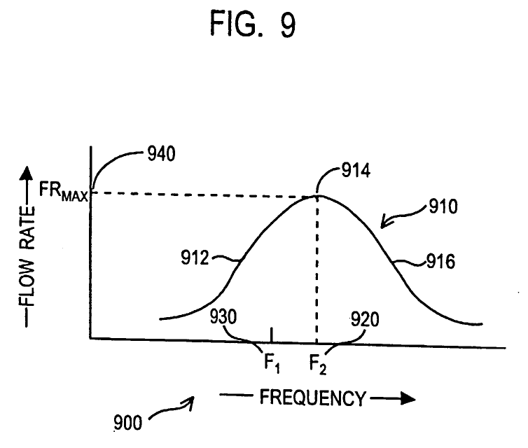

Referring now to FIG. 9, graph 900 includes curve 910. Curve 910 graphically depicts the flow rate of analyte in conduit 450 as a function of the frequency of a positive pressure assembly comprising an air pump. As those skilled in the art will appreciate, as a general matter, increasing the frequency of an air pump results in an increased air flow from that pump. Applicant has discovered, however, that when using his apparatus increasing the frequency of an air pump comprising positive pressure assembly 410 does not necessarily result in increasing the flow of analyte in conduit 440.

Curve 910 includes first portion 912, maximum flow rate portion 914, and third portion 916. A maximum analyte flow rate FR_{MAX} is obtained when operating the air pump at frequency F₂, i.e. point 920. When using frequencies less than frequency F₂, the analyte flow rate is less than FR_{MAX}. When using frequencies greater than frequency 920, the analyte flow rate of analyte is less than FR_{MAX}. Operating positive pressure assembly 410 at frequencies less than F₂, as the fluid flow rate through conduit 450 increases, the rate of release of analyte from wick 120 increases. Operating positive pressure assembly 410 at frequencies greater than frequency F₂, however, as the fluid flow rate through conduit 450 increases, the rate of release of analyte from wick 120 decreases.

In certain embodiments, Applicant's method includes operating an air pump comprising positive pressure assembly 410 at one frequency between F₁ and F₂ In certain embodiments, Applicant's method includes operating an air pump comprising positive pressure assembly 410 at a range of frequencies between F₁ and F₂, such that the frequency is initially set to F₁, and then continuously increased to F₂ to create a pressure wave. When the frequency of the air pump reaches F₂, the frequency is then dropped to F₁, and the process repeated.

In certain embodiments, frequency F₁ is about 90 percent the value of frequency F₂. In certain embodiments, frequency F₁ is about 80 percent the value of frequency F₂. In certain embodiments, frequency F₁ is about 75 percent the value of frequency F₂.

Feedback circuit 480 interconnects microprocessor 465 and valve 430. In certain embodiments, microprocessor 465 includes a computer useable medium having computer readable program code disposed therein to operate valve 430. In certain embodiments, apparatus 400 includes a computer program product usable with a programmable computer processor having computer readable program code embodied therein method to operate valve 430. Feedback circuit 490 interconnects microprocessor 465 and positive pressure assembly 410. In certain embodiments, microprocessor 465 includes a computer useable medium having computer readable program code disposed therein to operate assembly 410. In certain embodiments, apparatus 400 includes a computer program product usable with a programmable computer processor having computer readable program code embodied therein method to operate assembly 410.

In the event microprocessor 465 determines that an increased concentration of the one or more analytes disposed in analyte chamber 100/1000 is required, then microprocessor can adjust the flow rate of fluid supplied by assembly 410 in accord with FIG. 9, and/or increase the opening of valve 430. In the event microprocessor 465 determines that a decreased concentration of the one or more analytes disposed in analyte chamber 100/1000 is required, then microprocessor can adjust the flow rate of fluid supplied by assembly 410, and/or decrease the opening of valve 430.

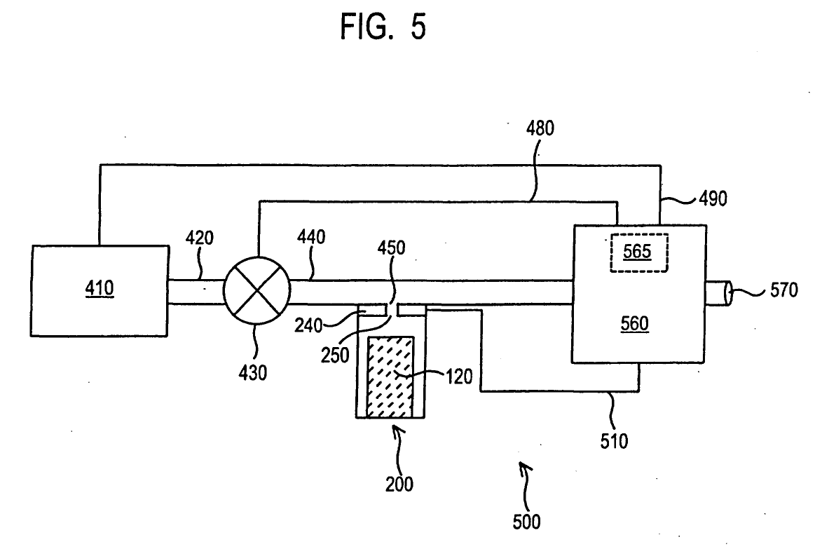

Referring now to FIG. 5, apparatus 500 includes elements 410, 420, 430, 440, 450, 480, and 490, as described above. Apparatus 500 further includes analyte chamber 200 (FIG. 2). Referring again to FIG. 2, analyte chamber 200 includes mechanical assembly 240 and orifice 250. Analyte chamber 200 releaseably attaches to air flow conduit 440 such that orifice 250 and orifice 450 communicate. Apparatus 500 further includes detector 560 microprocessor 565, and feedback circuits 480, 490, and 510.

In certain embodiments, detector 560 includes microprocessor 565. In certain embodiments, microprocessor 565 comprises computer hardware and software to operate detector 560, valve 430, positive pressure assembly 410, and mechanical assembly 240. In certain embodiments, microprocessor 565 comprises an Application Specific Integrated Circuit embodying program steps encoded into firmware to operate detector 560, valve 430, positive pressure assembly 410, and mechanical assembly 240. In the embodiment of FIG. 5, feedback circuit 480 interconnects valve 430 and microprocessor 565. In the embodiment of FIG. 5, feedback circuit 490 interconnects positive pressure assembly 410 and microprocessor 565. In the embodiment of FIG. 5, feedback circuit 510 interconnects mechanical assembly 240 and microprocessor 565.

In certain embodiments, microprocessor 565 includes a computer useable medium having computer readable program code disposed therein to operate valve 430. In certain embodiments, apparatus 500 includes a computer program product usable with a programmable computer processor having computer readable program code embodied therein method to operate valve 430. In certain embodiments, microprocessor 565 includes a computer useable medium having computer readable program code disposed therein to operate assembly 410. In certain embodiments, apparatus 500 includes a computer program product usable with a programmable computer processor having computer readable program code embodied therein method to operate assembly 410. In certain embodiments, microprocessor 565 includes a computer useable medium having computer readable program code disposed therein to operate mechanical assembly 240. In certain embodiments, apparatus 500 includes a computer program product usable with a programmable computer processor having computer readable program code embodied therein method to operate mechanical assembly 240.

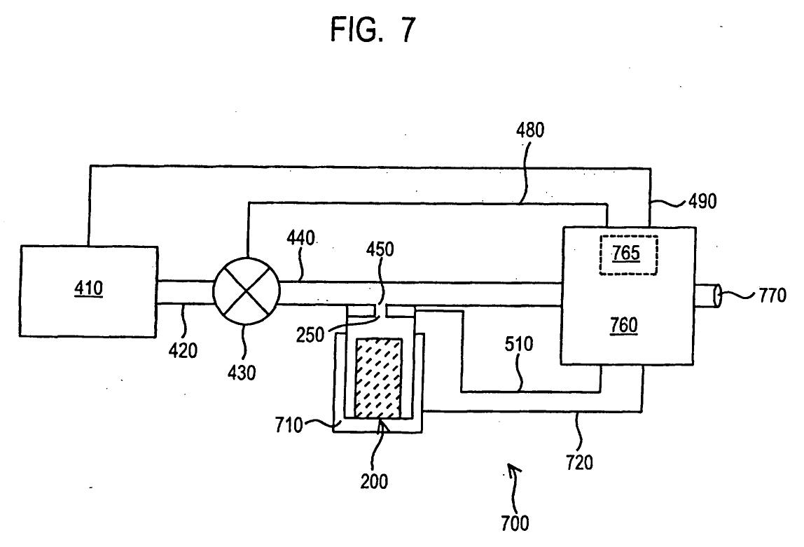

Referring now to FIG. 7, apparatus 700 includes elements 200, 250, 410, 420, 430, 440, 450, 480, 490, and 510, as described above. Apparatus 700 further includes detector 760, microprocessor 765, and heater 710. In the embodiment of FIG. 7, analyte chamber 200 is removeably disposed in heater 710.

In certain embodiments, detector 760 includes microprocessor 765. In certain embodiments, microprocessor 765 comprises computer hardware and software to operate detector 760, valve 430, positive pressure assembly 410, mechanical assembly 240, and heater 710. In certain embodiments, microprocessor 565 comprises an Application Specific Integrated Circuit embodying program steps encoded into firmware to operate detector 760, valve 430, positive air pressure assembly 410, mechanical assembly 240, and heater 710. In the embodiment of FIG. 7, feedback circuit 480 interconnects valve 430 and microprocessor 765. In the embodiment of FIG. 7, feedback circuit 490 interconnects positive pressure assembly 410 and microprocessor 765. In the embodiment of FIG. 5, feedback circuit 510 interconnects mechanical assembly 240 and microprocessor 765. In the embodiment of FIG. 5, feedback circuit 720 interconnects heater 710 and microprocessor 765.

In certain embodiments, microprocessor 765 includes a computer useable medium having computer readable program code disposed therein to operate valve 430. In certain embodiments, apparatus 700 includes a computer program product usable with a programmable computer processor having computer readable program code embodied therein method to operate valve 430. In certain embodiments, microprocessor 765 includes a computer useable medium having computer readable program code disposed therein to operate assembly 410. In certain embodiments, apparatus 700 includes a computer program product usable with a programmable computer processor having computer readable program code embodied therein method to operate assembly 410. In certain embodiments, microprocessor 765 includes a computer useable medium having computer readable program code disposed therein to operate mechanical assembly 240. In certain embodiments, apparatus 700 includes a computer program product usable with a programmable computer processor having computer readable program code embodied therein method to operate mechanical assembly 240.

In certain embodiments, microprocessor 765 includes a computer useable medium having computer readable program code disposed therein to operate heater 710. In certain embodiments, apparatus 700 includes a computer program product usable with a programmable computer processor having computer readable program code embodied therein method to operate heater 710.

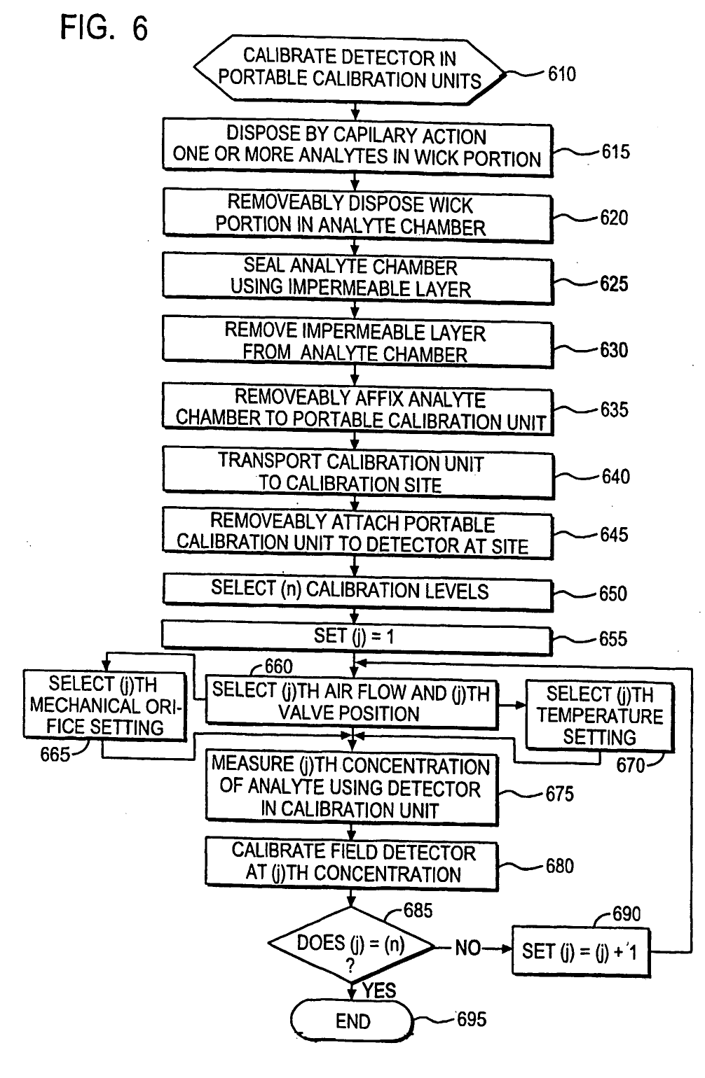

FIG. 6 summarizes the steps of Applicant's method, using Applicant's apparatus, to calibrate an immobile gas detector. Referring now to FIG. 6, in step 610 the detector in Applicant's portable calibration apparatus, such as detector 460 (FIG. 4)/560 (FIG. 5)/760 (FIG. 7), is calibrated for one or more analytes using known calibration standards.

In step 615, one or more analytes are disposed using capillary action in a wick assembly, such as wick 120. In step 620, the wick/analyte(s) combination is disposed in an analyte chamber, such as chamber 100 (FIG. 1)/200 (FIG. 2).

In embodiments using chamber 1000, in step 615 Applicants' wick and an ampoule containing the analyte, such as ampoule 1020, are disposed in housing 1010. In step 620, the ampoule is crushed thereby disposing the analyte in the wick by capillary action.

In step 625, the chamber containing the wick/analyte(s) combination is sealed. In certain embodiments, step 625 includes disposing a first layer, such as layer 140 (FIG. 1) over an open end of the analyte chamber, where that first layer is permeable to the one or more analytes disposed in the wick portion. In these embodiments, a second layer formed to include an orifice, such as layer 150 (FIG. 1) having orifice 160 (FIG. 1), is disposed over the first layer. In these embodiments, a third layer, such as layer 170, is disposed over the second layer, where the third layer is impermeable to the one or more analytes disposed in the wick portion.

In certain embodiments, step 625 includes disposing a mechanical orifice assembly, such as assembly 240, over an open end of the analyte chamber. In these embodiments, the mechanical orifice assembly is adjusted such that the orifice is closed.

In step 630, the outer impermeable outer layer, such as layer 170 (FIG. 1), is removed from the analyte chamber. In step 635, the analyte chamber is removeably affixed to the portable calibration unit, such as unit 400 (FIG. 4), 500 (FIG. 5), 700 (FIG. 7). In step 640, the portable calibration unit is transported to an immobile gas detector unit. In certain embodiments, step 640 precedes steps 630 and 635.

In step 645, Applicant's portable calibration apparatus is releaseably connected to the immobile gas detector. In step 650, the number (n) of different calibration concentrations for the one or more analytes are selected. In step 655, the (j)th calibration concentration is selected. In step 660 Applicants' method selects the (j)th fluid flow and the (j)th valve position. In certain embodiments, step 660 further includes generating by a microprocessor disposed in Applicant's portable calibration unit a first feedback signal and providing that first feedback signal to a positive fluid pressure assembly, such as assembly 410 (FIGS. 4, 5, 7). In certain embodiments, step 660 further includes generating by a microprocessor disposed in Applicant's portable calibration unit a second feedback signal and providing that second feedback signal to a valve assembly, such valve 430 (FIGS. 4, 5, 7).

In certain embodiments, Applicant's method transitions from step 660 to step 665. In certain embodiments, Applicant's method transitions from step 660 to step 670. In certain embodiments, Applicant's method transitions from step 660 to step 675. In certain embodiments, Applicant's method includes steps 665 and 670.

In step 665, Applicant's method selects the (j)th mechanical orifice setting, such as determining the size of orifice 250 (FIGS. 2, 5, 7) formed using mechanical assembly 240 (FIGS. 2, 5, 7). In these embodiments, step 665 further includes generating by a microprocessor disposed in Applicant's portable calibration unit a third feedback signal and providing that third feedback signal to the mechanical orifice. Applicant's method transitions from step 665 to step 675.

In step 670, Applicant's method selects the (j)th temperature setting a heater, such as heater 710 (FIG. 7). In these embodiments, step 670 further includes generating by a microprocessor disposed in Applicant's portable calibration unit a fourth feedback signal and providing that fourth feedback signal to the heater. Applicant's method transitions from step 670 to step 675.

In step 675, Applicant's method measures the (j)th concentration of one or more analytes provided from Applicant's portable calibration apparatus to the immobile gas detector. In step 680, Applicant's method calibrates the immobile gas detector using the measured concentration of step 675, i.e. the immobile detector is adjusted to recite the concentration of step 675. In step 685, Applicant's method determines if calibrations at each of the selected (n) calibration levels have been made, i.e. if (j) equals (n). If Applicant's method determines in step 685 that (j) equals (n), then Applicant's method transitions to step 6950 and ends. Alternatively, if Applicant's method determines in step 685 that (j) does not equal (n), then Applicant's method transitions from step 685 to step 690 wherein (j) is incremented. Applicant's method then transitions from step 690 to step 660 and continues.

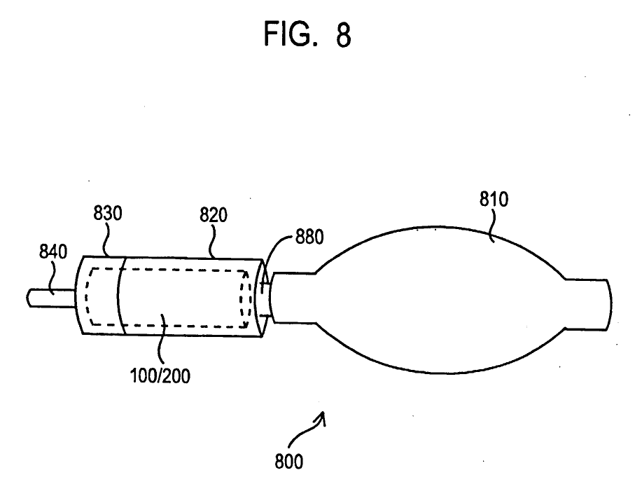

Referring now to FIG. 8, Applicant's invention further includes a portable apparatus 800 which comprises hand bulb 810, fluid flow conduit 880, housing 820, removeable end cap 830, and aperture 840. As those skilled in the art will appreciate, bulb 810 comprises a positive pressure fluid assembly. Squeezing bulb 810 provides a fluid, i.e. air, at a pressure greater than ambient atmospheric pressure. End cap 830 is removed from housing 820 and an analyte chamber, such as chamber 100/200/1000, is inserted into housing 820. Thereafter, end cap 830 is replaced. When using chamber 1000, ampoule 1020 is crushed and analyte 1030 is absorbed in wick 120 by capillary action. Thereafter, squeezing bulb 810 provides the analyte from aperture 840.

In certain embodiments, one or more individual steps of Applicants' method summarized in FIG. 6 may be combined, eliminated, or reordered.

In certain embodiments, Applicants' invention includes instructions residing in a microprocessor, such as microprocessor 465 (FIG. 4), 565 (FIG. 5), and/or 765 (FIG. 7), where those instructions are executed by microprocessor 465 (FIG. 4), 565 (FIG. 5), and/or 765 (FIG. 7), to performs steps 655, 660, 665, 670, and 675 recited in FIG. 6. In other embodiments, Applicants' invention includes instructions residing in any other computer program product, where those instructions are executed by a computer external to, or internal to, apparatus 400, 500, and/or 700, to perform steps 655, 660, 665, 670, and 675 recited in FIG. 6. In either case, the instructions may be encoded in an information storage medium comprising, for example, a magnetic information storage medium, an optical information storage medium, an electronic information storage medium, and the like. By “electronic storage media,” Applicants mean, for example, a device such as a PROM, EPROM, EEPROM, Flash PROM; compactflash, smartmedia, and the like.

While the preferred embodiments of the present invention have been illustrated in detail, it should be apparent that modifications and adaptations to those embodiments may occur to one skilled in the art without departing from the scope of the present invention as set forth in the following claims.

I claim:

1. An analyte chamber which can releaseably attached to a portable calibration unit, comprising:
a wick;
a liquid analyte absorbed in said wick;
a housing having an open end, wherein said wick is internally disposed within said housing;
a first layer disposed over and enclosing said open end;
headspace, wherein said headspace comprises the volume within said housing minus the volume of said wick;
gaseous analyte disposed in said headspace;
wherein said gaseous analyte is capable of passing through said first layer.

2. The analyte chamber of claim 1, wherein said analyte chamber cannot release said analyte in the liquid phase.

3. The analyte chamber of claim 1, further comprising a second layer disposed over said first layer, wherein said second layer comprises an orifice.

4. The analyte chamber of claim 3, wherein said orifice comprises a mechanical orifice.

5. The analyte chamber of claim 3, further comprising a plurality of analytes disposed in said wick.

6. A portable calibration apparatus, comprising:
a positive pressure assembly capable of providing a fluid at a pressure greater than atmospheric pressure;
a fluid flow conduit connected to said positive pressure assembly;
an analyte chamber disposed adjacent said fluid flow conduit, wherein said analyte chamber comprises a wick and a liquid analyte disposed in said wick, wherein said portable calibration apparatus cannot release said liquid analyte.

7. The portable calibration apparatus of claim 6, further comprising a detector connected to said fluid flow conduit.

8. The portable calibration apparatus of claim 6, wherein said analyte chamber further comprises:
a housing having an open end, wherein said wick and said analyte are internally disposed with said housing;
headspace, wherein said headspace comprises the volume within said housing minus the volume of said wick;
gaseous analyte disposed in said headspace;
a first layer disposed over and enclosing said open end, wherein said gaseous analyte is capable of passing through said first layer;
a second layer disposed over said first layer, wherein said second layer comprises a first orifice.

9. The portable calibration apparatus of claim 8, wherein said fluid conduit is formed to include a second orifice, and wherein said analyte chamber is releaseably attached to said fluid orifice such that said first orifice communicates with said second orifice.

10. The portable calibration apparatus of claim 9, further comprising:
a microprocessor; and
a first feedback circuit interconnecting said microprocessor and said positive pressure assembly.

11. The portable calibration apparatus of claim 10, wherein said fluid conduit comprises a first portion and a second portion, further comprising:
a valve interconnecting said first portion of said fluid conduit and said second portion of said fluid conduit;
a second feedback circuit interconnecting said microprocessor and said valve.

12. The portable calibration apparatus of claim 11, wherein said first orifice comprises an electromechanical orifice, further comprising a third feedback circuit interconnecting said microprocessor and said electromechanical orifice.

13. The portable calibration apparatus of claim 12, further comprising:
a heater, wherein said analyte chamber is capable of being removeably disposed in said heater;
a fourth feedback circuit interconnecting said microprocessor and said heater.

14. A method to calibrate a stationary gas detector, comprising the steps of:
providing a portable calibration apparatus comprising a portable detector and an analyte chamber comprising a wick;
disposing by capillary action a liquid analyte in said wick, such that all of said analyte is absorbed in said wick, and such that said wick cannot release said analyte in the liquid phase;
providing a concentration of said analyte in the gaseous phase to said portable detector;
measuring said concentration using said portable detector;
providing said gaseous analyte to said stationary detector;
calibrating said stationary detector using said concentration.

15. The method of claim 14, further comprising the steps of:
establishing (n) calibration levels;
setting (i) to 1;
providing the (i)th concentration of said gaseous analyte;
measuring said (i)th concentration using said portable detector;
providing said (i)th concentration of said gaseous analyte to said stationary detector;
calibrating said stationary detector using said (i)th concentration;
determining if (i) equals (n);
operative if (i) is less than (n), incrementing (i) and repeating said providing, measuring, calibrating, and determining steps;
operative if (i) equals (n), ending the calibration process.

16. The method of claim 14, wherein said detector further comprises a microprocessor, and wherein said portable calibration unit further comprises a positive pressure fluid assembly and a feedback circuit interconnecting said microprocessor and said positive pressure fluid assembly, said method further comprising the step of adjusting the fluid flow provided by said positive pressure fluid assembly.

17. The method of claim 14, wherein said analyte chamber further comprises a mechanical orifice, and wherein said portable calibration unit further comprises a feedback circuit interconnecting said microprocessor and said mechanical orifice, said method further comprising the step of adjusting said mechanical orifice.

18. The method of claim 14, wherein said portable calibration unit further comprises a heater and a feedback circuit interconnecting said microprocessor and said heater, wherein said analyte chamber can be removeably disposed in said heater, said method further comprising the step of adjusting the temperature of said heater.

19. An article of manufacture comprising a computer useable medium having computer readable program code disposed therein to adjust the available concentration of a gaseous analyte, wherein said article of manufacture comprises a portable detector, an analyte chamber comprising a wick and a liquid analyte disposed in said wick, and a positive pressure fluid assembly, wherein said article of manufacture is capable of providing a concentration of said analyte in the gas phase to said portable detector, the computer readable program code comprising a series of computer readable program steps to effect:
measuring a first concentration of said gaseous analyte emitted from said wick;
adjusting the flow rate of fluid provided by said positive pressure fluid assembly;
measuring a second concentration of said gaseous analyte.

20. The article of manufacture of claim 19, wherein said analyte chamber further comprises a mechanical orifice, said computer readable program code further comprising a series of computer readable program steps to effect adjusting the size of said mechanical orifice.

21. The article of manufacture of claim 20, further comprising a heater, wherein said analyte chamber can be removeably disposed in said heater, said computer readable program code further comprising a series of computer readable program steps to effect adjusting the temperature of said heater.
